(19) United States
(12) Patent Application Publication
(10) Pub. No.: US 2004/0235154 A1
(43) Pub. Date: Nov. 25, 2004

(54) Polymerase chain reaction device and method of regulating opening and closing of inlet and outlet of the polymerase chain reaction device

(76) Inventors: Kwang-wook Oh, Gyeonggi-do (KR); Dae-sung Yoon, Seoul (KR); Young-sun Lee, Gyeonggi-do (KR); Sun-hee Kim, Gyeonggi-do (KR); Geun-bae Lim, Gyeonggi-do (KR); Young-a Kim, Gyeonggi-do (KR); Jung-im Han, Seoul (KR)

Correspondence Address: Michael A. Cantor, 55 Griffin South Road, Bloomfield, CT, 06002, US

(21) Appl. No.: 10/783,127

(22) Filed: Feb. 19, 2004

(30) Foreign Application Priority Data
Feb. 20, 2003 (KR) 2003-10729

(51) Int. Cl. C12M 1/34

(52) U.S. Cl. 435/303.1; 435/288.5; 435/297.2

(57) ABSTRACT

A polymerase chain reaction (PCR) device with micro-valves, which are opened or closed using a simple control mechanism is provided. The PCR device includes: an inlet through which a biochemical fluid is injected; an outlet through which the biochemical fluid is discharged; a PCR channel positioned between the inlet and the outlet; first and second micro-valves, which control opening and closing of the inlet and the outlet; and a sol-gel transformable material, which transforms from a sol state into a gel state at a temperature lower than DNA denaturation temperature, annealing temperature and extension temperature and higher than room temperature.

### BACKGROUND OF THE INVENTION

This application claims the priority of Korean Patent Application No. 2003-10729, filed on Feb. 20, 2003, in the Korean Intellectual Property Office, the disclosure of which is incorporated herein in its entirety by reference.

1. Field of the Invention

The present invention relates to a polymerase chain reaction (PCR) device, and more particularly, to a PCR device with an inlet and an outlet, which are opened or closed using a single control mechanism, and a method of regulating opening or closing of the inlet and the outlet of the PCR device.

2. Description of the Related Art

In PCR devices repetitive heating and cooling to denaturation temperature, annealing temperature, and extension temperature are performed to amplify nucleic acids. The internal pressure of the PCR device rises while a biochemical sample is repeatedly heated and cooled to denature, anneal, and extend DNA, so that the biochemical sample may evaporate or flow out of the PCR device during reaction. Therefore, it is required to control the PCR device to prevent the biochemical sample from evaporating or flowing out of the device during reaction.

Most micro-lab-on-chips include a nucleic acid extraction device, a PCR device, and a nucleic acid detection device. The nucleic acid extraction device is connected to an inlet of the PCR device, and the nucleic acid detection device is connected to an outlet of the PCR device. To prevent a biological sample from evaporating or flowing out of the PCR device of a micro-lab-on-a-chip, caused due to a rise in internal pressure resulting from repeated heating and cooling performed to amplify nucleic acids, positioning valves near the inlet and outlet of the PCR device has been suggested.

U.S. Pat. No. 6,168,948 B1 discloses a PCR device with a pneumatic valve near the inlet thereof and a gas permeable valve near the outlet thereof, and a nucleic acid extraction device with a pump for applying compression force to the pneumatic valve. However, it is complicated to previously form a flexible valve membrane and a hydrophobic valve membrane near the inlet and outlet of the PCR device. Furthermore, an additional system such as a pump is required to cause these membranes to function, thereby making it difficult to miniaturize the entire system.

The above-identified patent also discloses use of a diaphragm valve. However, the diaphragm valve has a complicated, multi-layered structure and a diaphragm, and an external force must be applied to deflect the diaphragm valve using electromagnetic, thermal, pneumatic, piezoelectric, electrostatic actuations, etc.

U.S. Pat. No. 6,130,098 discloses a method of regulating flow of fluid by treating an internal surface of a micro-channel to be hydrophilic or hydrophobic. However, this method is unsuitable for PCR devices. The internal surface of the micro-channel treated to be hydrophobic causes biochemical fluid vaporizing at a PCR temperature to easily leak from the micro-channel.

D. J. Beebe, J. S. Moore, Q. Yu, R. H. Liu, M. L. Kraft, B. H. Jo, and C. Devadoss disclosed use of a polymeric material in the manufacture of a valve structure using light (“Microfluidic tectonics: A comprehensive construction of platform for microfluidic systems”, PNAS, Dec. 5, 2000, Vol. 97, No. 25, p. 13493). Although the valve structure and a micro-channel structure can be manufactured easily using the polymeric material, the valve functions properly only with a specific chemical substance, so that its use for PCR devices is limited, depending on chemical substance.

Y. Liu, C. B. Rauch, R. L. Stevens, R. Lenigk, J. Yang, D. B. Rhine, and P. Grodzinski, disclosed use of a pluronic gel, which is in crystalline form at room temperature, for a valve in a PCR device. The pluronic gel, a polymeric substance that changes phase depending on temperature, exists in crystalline form at room temperature, and its viscosity greatly decreases at 5. or less. Therefore, the pluronic gel can function as a valve. However, a cooler such as a Peltier thermoelectric device is required to drop the temperature to 5. or less to lower the viscosity of the pluronic gel and operate it as a valve.

Japanese Patent Publication No. 2003-163022 discloses a method of controlling flow of fluid in a micro-channel in a micro-system by injecting a sol-gel transformable material into the micro-channel and applying a stimulus to a local region of the micro-channel to gelate the fluid. Heat or a voltage is applied as a stimulus to induce gelation of the fluid and regulate flow of the fluid.

U.S. Pat. No. 6,382,254 B1 discloses a micro-valve device including a micro-channel, a heater placed in contact with at least a region of the micro-channel, and a carrier liquid containing a sol-gel transformable substance, which raises the viscosity of liquid flowing along the micro-channel when heated by the heater. In other words, the additional heater is required to regulate flow of the liquid with the micro-valve device.

### SUMMARY OF THE INVENTION

The present invention provides a polymerase chain reaction (PCR) device with a micro-valve capable of opening and closing an inlet and an outlet of the PCR device without requiring an additional [heater] heat source.

The present invention also provides an easy method of regulating opening and closing the inlet and the outlet of the PCR device.

According to an aspect of the present invention, there is provided a PCR device comprising: an inlet through which a biochemical fluid is injected; an outlet through which the biochemical fluid is discharged; a PCR channel positioned between the inlet and the outlet; first and second micro-valves, which control opening and closing of the inlet and the outlet; and a sol-gel transformable material, which transforms from a sol state into a gel state at a temperature lower than DNA denaturation temperature, annealing temperature and extension temperature and higher than room temperature.

The first and second micro-valves containing the sol-gel transformable material may have any shape provided that they can control opening and closing of the inlet and outlet of the PCR device.

According to another aspect of the present invention, there is provided a method of regulating opening and closing of an inlet and an outlet of a PCR device, the method comprising: connecting micro-valves, each of which contains a sol-gel transformable material that transforms from a sol state to a gel state at a temperature lower than DNA denaturation temperature, annealing temperature and extension temperature regarding PCR and higher than room temperature, to the inlet and the outlet of the PCR device; and inducing a sol-to-gel transformation in the micro-valves using temperature variations in a thermal cycle of PCR.

The sol-gel transformable material contained in the micro-valve according to the present invention transforms into a gel state at a temperature lower than DNA denaturation temperature, annealing temperature and extension temperature and higher than room temperature, so that it can function properly as a valve spontaneously during PCR. Therefore, no additional [heater] heat source is required to operate the micro-valve. In addition, since the sol-gel transformable material transforms back into a sol state at a temperature higher than room temperature, no additional cooler is required to allow biochemical fluid to be discharged through the outlet of the PCR device. Any sol-gel transformable material may be used for the micro-valve according to the present invention. A representative example of the sol-gel transformable material includes methyl cellulose.

### BRIEF DESCRIPTION OF THE DRAWINGS

The above and other features and advantages of the present invention will become more apparent by describing in detail exemplary embodiments thereof with reference to the attached drawings in which:

FIG. 1 illustrates a lab-on-a-chip including a polymerase chain reaction (PCR) device equipped with micro-valves according to an embodiment of the present invention;

FIG. 2 is a diagram for explaining the operational principle of the micro-valves in the PCR device in FIG. 1;

FIG. 3 is a graph illustrating the relationship between a gelation temperature of a sol-gel transformable material used for the micro-valves according to the present invention, DNA denaturation temperature, annealing temperature, and extension temperature;

FIG. 4 illustrates a PCR device with micro-valves according to another embodiment of the present invention;

FIG. 5 illustrates an operational principle of the micro-valves in the PCR device in FIG. 4;

FIG. 6 illustrates a PCR device with micro-valves according to another embodiment of the present invention;

FIG. 7 illustrates an operational principle of the micro-valves in the PCR device in FIG. 6;

FIGS. 8 and 9 illustrate PCR devices with micro-valves according to other embodiments of the present invention;

FIG. 10 is a graph of shear force versus temperature obtained using various concentrations of methyl cellulose solutions;

FIG. 11 is a graph of absorbance versus temperature obtained using various concentrations of methyl cellulose solutions;

FIG. 12 illustrates the ¹H NMR absorption spectra of a sample containing 0.5% methyl cellulose and 2% NaCl at 25., 35., 45., and 60.;

FIG. 13 is a photograph of the results of electrophoresis performed using the PCR products from a sample containing 0.5% methyl cellulose; and

FIG. 14 is a graph of the results of electrophoresis performed using the PCR products amplified using a micro-PCR chip according to an embodiment of the present invention

### DETAILED DESCRIPTION OF THE INVENTION

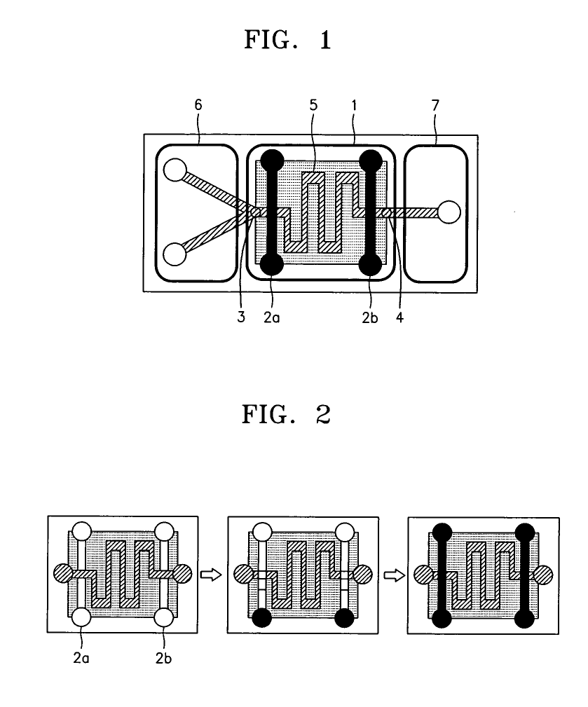

A lab-on-a-chip including a polymerase chain reaction (PCR) device according to an embodiment of the present invention is illustrated in FIG. 1. Referring to FIG. 1, a PCR device 1 according to an embodiment of the present invention includes an inlet 3, an outlet 4, a PCR channel 5 connected between the inlet 3 and the outlet 4, a first micro-valve 2a and a second micro-valve 2b, which controls opening and closing of the inlet 3 and the outlet 4, respectively, and a [heater] heat source (not shown) for operating the PCR device 1 and the first and second micro-vales 2a and 2b. A nucleic acid extraction device 6 and a nucleic acid detection device 7 are connected to the inlet 3 and the outlet 4 of the PCR device 1, respectively.

The first and second micro-valves 2a and 2b of the PCR device 1 contain a sol-gel transformable material, which changes phase into a gel state at a temperature that is lower than denaturation temperature, annealing temperature and extension temperature in a PCR of DNA and is higher than room temperature.

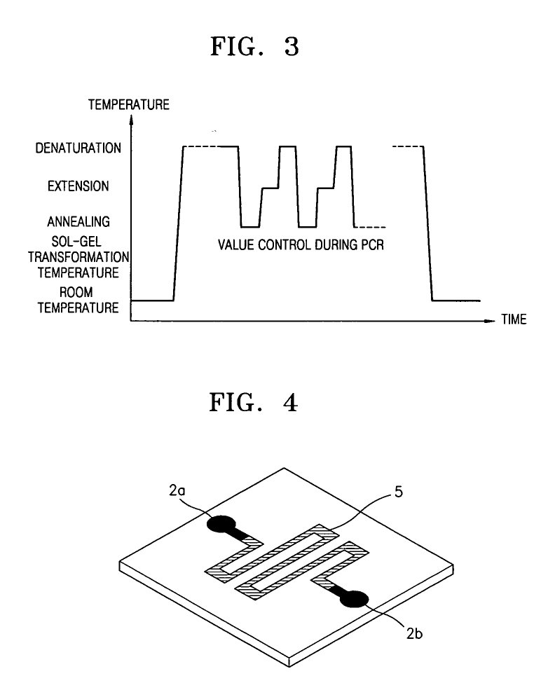

The relationship between the gelation temperature of the sol-gel transformable material, the DNA denaturation temperature, the annealing temperature, and the extension temperature is illustrated in FIG. 3. Since the gelation temperature of the sol-gel transformable material used in the present invention is lower than the PCR temperatures and higher than room temperature, the sol-gel transformable material can control opening and closing of the micro-valve when PCR is performed. The sol-gel transformable material is present in a sol state before PCR, i.e., at room temperature, and transforms into a gel state by gelation when heated to a PCR temperature by the heater connected to the PCR device. When the sol-gel transformable material contained in the first and second micro-valves 2a and 2b transforms from a sol state to a gel state, it is prevented that biochemical fluid flows or evaporates out of the PCR device. When PCR is completed and the temperature of the PCR device 1 drops to room temperature, the sol-gel transformable material transforms from a gel state to a sol state to allow the biochemical fluid in the PCR device 1 to flow toward the outlet 4 or the nucleic acid detection device 7.

A non-limiting example of the sol-gel transformable material used for the first and second micro-valves 2a and 2b of the PCR device 1 according to the present invention includes methyl cellulose. Since a gelation temperature of methyl cellulose lies between a PCR temperature and room temperature, the methyl cellulose can properly function as a micro-valve when PCR starts, without requiring an additional heater or cooler.

Resistance heat generated by flowing a current through a thin metal plate made of, for example, platinum (Pt), aluminum (Al), copper (Cu), etc. may be used to heat the temperature of the PCR device 1 to a DNA denaturation temperature, an annealing temperature and an extension temperature and operate the first and second micro-valves 2a and 2b. Other examples of the heat source include, but are not limited to, by a Peltier thermoelectric device, an infra-red light, a laser light, an electromagnetic thermal device, an AC voltage, and the like.

As illustrated in FIG. 1, the PCR device 1 according to the present invention may be used in connection with the nucleic acid extraction device 6 and the nucleic acid detection device 7. The nucleic acid extraction device 6 extracts a target nucleic acid from a sample, the PCR device 1 amplifies the extracted target nucleic acid, and the nucleic acid detection device 7 identifies the amplified nucleic acid.

FIG. 2 is a diagram for explaining the operational principle of the first and second micro-valves 2a and 2b of the PCR device 1 in FIG. 1. A predetermined amount of a biochemical fluid is injected into the inlet 3 of the PCR device 1. When the biochemical fluid enters and fills the PCR channel 5, a sol-gel transformable material is injected into the first and second micro-valves 2a and 2b, and a thermal cycler is operated to initiate a PCR. The sol-gel transformable material in the first and second micro-valves 2a and 2b gelates simultaneously with the PCR reaction so that the biochemical fluid is kept in the PCR channel 5. As a result, the biochemical fluid does not flow or evaporate out of the PCR channel 5, so that there is no drop in the yield of the PCR.

The first and second micro-valves 2a and 2b according to the present invention are positioned near the inlet 2 and the outlet 4 of the PCR device 1. The first and second micro-valves 2a and 2b may be formed in any shape provided that they can function as micro-valves at the inlet 3 and the outlet 4. As illustrated in FIG. 1, the first and second micro-valves 2a and 2b according to the present invention may be formed as channels intersecting the inlet 3 and the outlet 4 of the PCR device 1.

Examples of micro-valves according to the present invention, which have various shapes, are illustrated in FIGS. 4 through 9.

FIG. 4 illustrates a PCR device with first and second micro-valves 2a and 2b, which correspond to the inlet 3 and the outlet 4 of the PCR device, respectively. Since the first and second micro-valves 2a and 2b are integrated with the inlet 3 and the outlet 4 of the PCR device, it is unnecessary to form a separate micro-valve structure. As a modified example, the first and second micro-valves 2a and 2b may be extended from the inlet 3 and the outlet 4 of the PCR device in a direction in which biochemical fluid is injected or discharged.

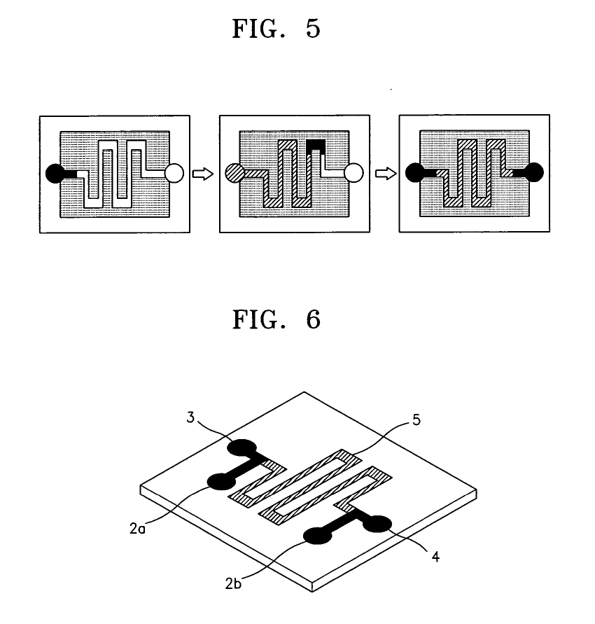

The first and second micro-valves 2a and 2b illustrated in FIG. 4 operate according to the principle illustrated in FIG. 5. Referring to FIG. 5, initially a sol-gel transformable material is injected into the inlet of the PCR device. Next, a biochemical fluid containing a target nucleic acid to be amplified is injected, and the sol-gel transformable material is injected once more such that the sol-gel transformable material reaches the inlet and the outlet. The sol-gel transformable material in the inlet and the outlet gelates at the start of PCR and clogs the inlet and outlet of the PCR device.

FIG. 6 illustrates a PCR device with first and second micro-valves 2a and 2b, which are interconnected with the inlet and the outlet of the PCR device and branch off from portions of the PCR channel near the inlet and the outlet in a different direction from a direction in which biochemical fluid is injected and discharged.

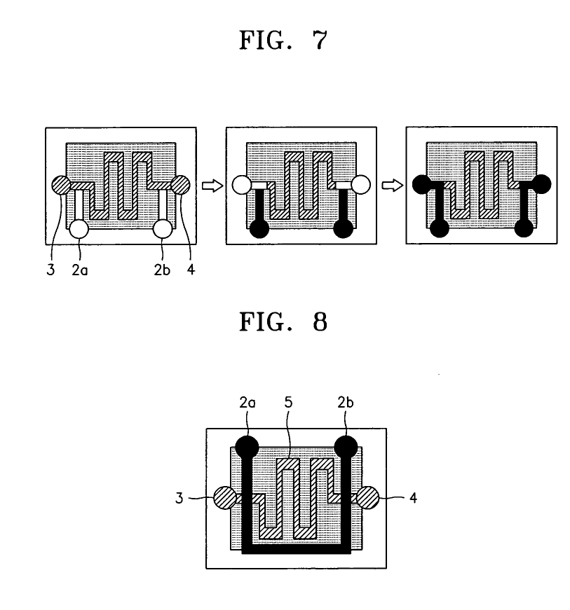

The first and second micro-valves 2a and 2b illustrated in FIG. 6 operate according to the principle illustrated in FIG. 7. Referring to FIG. 7, the PCR channel 5 is filled with biochemical fluid containing a target nucleic acid to be amplified, and a sol-gel transformable material is injected into the first and second micro-valves [2] 2aand 2b such that it reaches the inlet 3 and the outlet 4 of the PCR device. The sol-gel transformable material gelates at the start of PCR and clogs the inlet and the outlet so that the initially injected biochemical fluid can be fully amplified without evaporating or flowing out of the PCR device. When the temperature of the PCR device drops to room temperature after PCR is terminated, the sol-gel transformable material transforms into a sol state to open the inlet and the outlet and allow the biochemical fluid to flow through the outlet of the PCR device.

FIG. 8 illustrates a PCR device with first and second micro-valves 2a and 2b, which intersect portions of the PCR channel 5 near the inlet 3 and the outlet 4 of the PCR device and are interconnected at one end.

The first and second micro-valves 2a and 2b illustrated in FIG. 8 operate as follows. Initially, the PCR channel 5 is filled with a biochemical fluid containing a target nucleic acid to be amplified, and a sol-gel transformable material is injected into at least one of the first and second micro-valves 2a and 2b to fill the first and second micro-valves 2a and 2b. The sol-gel transformable material gelates at the start of PCR and blocks the biochemical fluid from flowing toward the inlet 3 and the outlet 4 so that the initially injected biochemical fluid can be fully amplified in the PCR channel 5, without evaporating or flowing out of the PCR device. When the temperature of the PCR device drops to room temperature after PCR is terminated, the sol-gel transformable material transforms into a sol state to open the inlet 3 and the outlet 4 and allow the biochemical fluid to flow through the outlet of the PCR device.

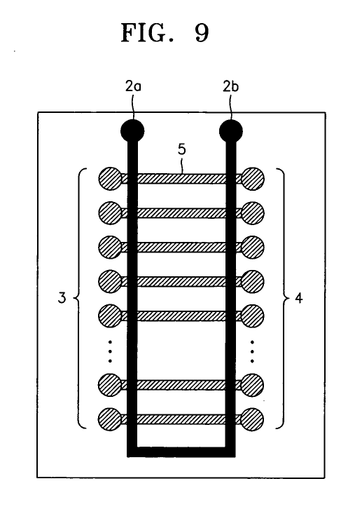

FIG. 9 Illustrates a plurality of PCR devices with common first and second micro-valves 2a and 2b, which intersect portions of each PCR channel 5 near the inlet 3 and the outlet 4 of each of the PCR devices and are interconnected at one end. In a lab-on-a-chip including a plurality of PCR devices, the plurality of PCR devices may be interconnected by micro-valves laid near the inlets and outlets of the PCR devices, as illustrated in FIG. 9, the micro-valves being interconnected at one end. A sol-gel transformable material is injected into the interconnected micro-valves, which intersect the PCR channels of the PCR devices near the inlets and outlets, to simultaneously control opening and closing of the PCR devices.

The present invention will be described in greater detail with reference to the following examples. The following examples are for illustrative purposes and are not intended to limit the scope of the invention.

### EXAMPLE 1

### Reversible Sol-Gel Transformation of Methyl Cellulose

Experimental-grade methyl cellulose powder was purchased from Aldrich Chemicals Co. The methyl cellulose used had a viscosity of 400 cP (2%) at room temperature, an average molecular weight of 130,000, a polydispersity of 1.8, and an methyl radical substitution of 2.1. Portions of the methyl cellulose powder were dissolved in 4. deionized water and left for 24 hours to obtain fully dissolved 0.5%, 1.2%, 1.5%, and 2.0% by weight methyl cellulose solutions.

Rheological properties depending on temperature and concentration were measured using the methyl cellulose solutions. Variations in shear force were measured using a stress control viscometer (Carrimed CS50). Gelation temperature was measured by measuring an absorption spectrum at 700 nm using a multi-spect 1501 UV/VIS spectrophotometer (Shimadzu). The gelation of the methyl cellulose solutions is attributed to phase separation, which affects turbidity of the methyl cellulose solution.

Temperature-dependent rheological properties of the methyl cellulose solutions were measured at various temperatures to determine whether the methyl cellulose solutions could function as a gel valve.

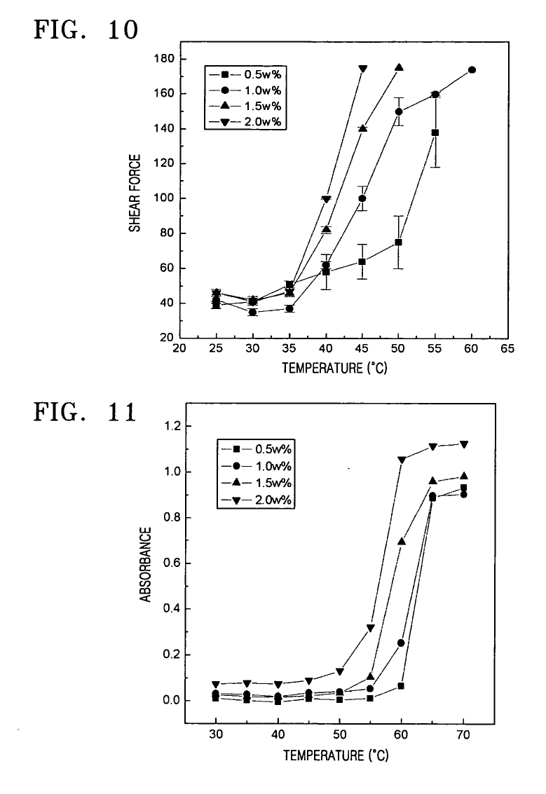

The results of measuring variations in shear force at various temperatures and various concentrations using the stress control viscometer (Carrimed CS50) are shown in FIG. 10. As is apparent from FIG. 10, the shear force increased in all of the methyl cellulose solutions as temperatures increased, and gelation started near 35. At 35., the shear force increased sharply in the 1.0% or greater by weight methyl cellulose solutions but slowly in the 0.5% by weight methyl cellulose solution.

Absorption spectra at 700 nm were measured using the methyl cellulose solutions while varying temperature. The results are shown in FIG. 11. Referring to FIG. 11, it is inferred from a sharp increase in absorbance at a temperature higher than 55. that the gelation temperatures of the methyl cellulose solutions are near 55. Comparing the results of the shear force measurement in FIG. 10 and the results of the optical absorbance measurement in FIG. 11, there is a large discrepancy in inferred gelation temperatures. 0.5% by weight methyl cellulose solution had a difference in gelation temperature of about 20. between the results of FIGS. 10 and 11.

The absorbance of all of the solutions remained constant in a temperature range between 35. and 55. while the viscosity continued to increase in that temperature range. As is apparent from the results in FIG. 11, methyl cellulose changes phase from a transparent sol to a transparent gel and then an opaque gel with rising temperatures. However, the optical measurements failed to provide an accurate gelation temperature of methyl cellulose. Meanwhile, the results of the shear force measurement support the possibility that suitable gel valve materials can be screened by measuring such a rheological property of the material.

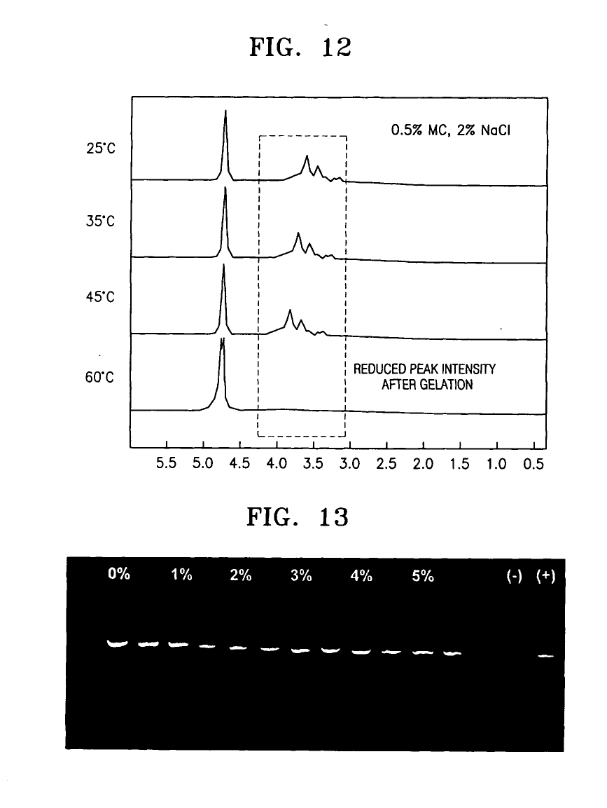

In another rheological property experiment, temperature-dependant variations in chemical bonds of methyl cellulose were measured by NMR. ¹H NMR was measured using a 0.5% methyl cellulose solution containing 2% NaCl at 25., 35., 45., and 60. The resulting ¹H NMR spectra are shown in FIG. 12.

Reduced peak intensities were observed at temperatures higher than 35. This result implies that the fluidity of the polymer chain gradually deteriorates with rising temperature. The reduction in peak intensity is attributed to the substitution of methyl groups by protons.

### EXAMPLE 2

A PCR solution containing a 10×PCR buffer (750 mM Tris-HCl (pH 9.0), 150 mM (NH₄)₂SO₄, 25 mM MgCl₂, 1 mg/ml BSA), 250 μ M dNTP, 500 nM upper primer (5′-cccttgctgagcagatcccgtc-3′), 500 nM lower primer (5′-gggatggtgaagcttccagcc-3′), 500 ng of the human genome DNA, and 4.8 of Taq DNA polymerase (a molar ratio of 28:1 (TaqStartAb: Taq DNA polymerase)) was prepared.

A 0.5% methyl cellulose solution was added to the PCR solution in a volume ratio of 0-0.05:1. Amplification was carried out using 100 μl of the PCR solution in a DNA thermal cycler (Eppendorf Co.). After incubation at 95. for 3 minutes, 40 cycles of reaction at 95. for 30 seconds, at 55. for 15 seconds, and at 72. for 1 minute were carried out and followed by a final reaction at 72. for 3 minutes. The amplified PCR products were quantized. The results are shown in Table 1.

TABLE 1

| % of Methyl | | |
|---|---|---|
| Cellulose | Weight (ng) | Average Weight (ng) |
| 0% | 168.152 | 170.22 |
| 0% | 172.288 | |
| 1% | 185.175 | 172.1665 |
| 1% | 159.158 | |
| 2% | 172.086 | 170.687 |
| 2% | 169.288 | |
| 3% | 178.968 | 174.2535 |
| 3% | 169.539 | |
| 4% | 177.369 | 175.1705 |
| 4% | 172.972 | |
| 5% | 167.588 | 167.6405 |
| 5% | 167.693 | |
| N | −0.188 | |
| P | 160.369 | 160.369 |

N: negative control group (containing 0.5% methyl cellulose and no human genome DNA);
P: positive control group (containing human genome DNA and no 0.5% methyl cellulose)

The amplified PCR products were analyzed by electrophoresis on 2% TAE agarose gel. A photograph of the resolved bands of the PCR products to which 0.5% methyl cellulose was added, separated by electrophoresis at UV 305 nm, is shown in FIG. 13.

As is apparent from the quantitative data of the PCR products, which contained methyl cellulose in the range of 0-5%, in Table 1, a 5% or less methyl cellulose solution does not affect PCR results.

### EXAMPLE 3

### Micro-PCR Chip

DNA amplification was carried out using the sol-gel micro-valve according to the present invention. A micro-chip manufactured by binding a silicon substrate and a glass substrate was used as a PCR chip. The micro-chip had a 1 μl-channel in the silicon substrate, and a platinum heater was installed on an external surface of the silicon substrate aligned with the silicon channel. An inlet and an outlet were formed in the glass substrate. The sample prepared in Example 2 was used. A 0.5% methyl cellulose solution was used as a sol-gel transformable material.

1 μl of the 0.5% methyl cellulose solution, 1 μl of the sample, and 1 μl of the 0.5% methyl cellulose solution were sequentially injected into a capillary tube. This capillary tube was connected to the inlet of the PCR chip such that the solutions flowed toward the channel. Next, power was applied to the heater to initiate DNA amplification.

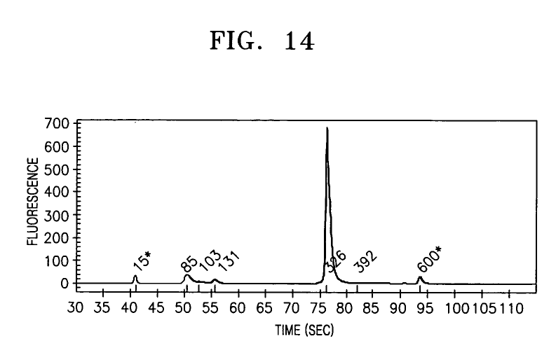

PCR was carried out using the following temperature profile: incubation at 95. for 3 minutes, 40 cycles of reaction at 95. for 30 seconds, at 55. for 15 seconds, and at 72. for 1 minute, and a final reaction at 72. for 3 minutes. The amplified PCR products were analyzed by electrophoresis using an Agilent Bioanalyzer 2001. The results are shown in FIG. 14. As can be inferred from the results in FIG. 14, the PCR chip with the sol-gel micro-valve according to the present invention can be used for DNA amplification.

It was also confirmed through the experiments that the biochemical fluid containing DNA does not evaporate and flow out of the PCR channel during PCR.

As described above, an inlet and an outlet of a PCR device can be simply opened or closed using a sol-gel micro-valve according to the present invention, without requiring an additional heat source. Due to the structural simplicity of the micro-valve, PCR devices can be easily mounted on a micro-chip, such as a lab-on-a-chip, when using the micro-valve according to the present invention, based on micro-processing technology applied to silicon, glass, polymers, etc. The PCR device can be miniaturized to be portable.

In addition, the micro-valve according to the present invention prevents biological fluid in the PCR device from evaporating or flowing out of the device, thereby enabling DNA amplification using a constant amount of biochemical fluid. Compared with conventional complicated micro-metering systems frequently used even when handing a trace of biochemical fluid on the order of microliters and picoliters, the micro-valve according to the present invention can be simply operated by just injecting a sol-gel transformable material to inlet and outlet regions of a PCR channel. Furthermore, the injection of the sol-gel transformable material allows an accurate amount of biochemical fluid to be injected into the PCR device, as well as prevents evaporation of the biological fluid to be amplified. The injection of an accurate amount of biochemical sample prevents waste of the biochemical fluid.

In addition, the micro-valve according to the present invention initiates its operation spontaneously at the start of amplification and terminates its function as a valve when the amplification finishes, thereby enabling a rapid transfer of the biochemical fluid for a subsequent process.

While the present invention has been particularly shown and described with reference to exemplary embodiments thereof, it will be understood by those of ordinary skill in the art that various changes in form and details may be made therein without departing from the spirit and scope of the present invention as defined by the following claims.

1. A PCR (polymerase chain reaction) device comprising:
an inlet through which a biochemical fluid is injected;
an outlet through which the biochemical fluid is discharged;
a PCR channel positioned between the inlet and the outlet;
first and second micro-valves, which control opening and closing of the inlet and the outlet; and
a sol-gel transformable material, which transforms from a sol state into a gel state at a temperature lower than DNA denaturation temperature, annealing temperature and extension temperature and higher than room temperature.

2. The PCR device of claim 1, wherein the sol-gel transformable material is methyl cellulose.

3. The PCR device of claim 1, wherein the first and second micro-valves form the inlet and outlet of the PCR device, respectively.

4. The PCR device of claim 1, wherein the first micro-valve extends in a direction in which the biochemical fluid is injected into the inlet, and the second micro-valve extends in a direction in which the biochemical fluid is discharged through the outlet.

5. The PCR device of claim 1, wherein the first and second micro-valves are interconnected with the inlet and the outlet, respectively, the first micro-valve branches off from a portion of the PCR channel near the inlet in a different direction from a direction in which the biochemical fluid is injected, and the second micro-valve branches off from a portion of the PCR channel near the outlet in a different direction from a direction in which the biochemical fluid is discharged.

6. The PCR device of claim 1, wherein the first and second micro-valves intersect portions of the PCR channel near the inlet and the outlet of the PCR device, respectively.

7. The PCR device of claim 6, wherein one end of the first micro-valve is connected to one end of the second micro-valve.

8. The PCR device of claim 1, wherein the first and second micro-valves intersect portions of PCR channels of a plurality of PCR devices near inlets and outlets of the PCR devices, respectively.

9. The PCR device of claim 8, wherein one end of the first micro-valve is connected to one end of the second micro-valve.

10. A method of regulating opening and closing of an inlet and an outlet of a PCR device, the method comprising:
connecting micro-valves, each of which contains a sol-gel transformable material that transforms from a sol state to a gel state at a temperature lower than DNA denaturation temperature, annealing temperature and extension temperature regarding PCR and higher than room temperature, to the inlet and the outlet of the PCR device; and
inducing a sol-to-gel transformation in the micro-valves using temperature variations in a thermal cycle of PCR.

11. The method of claim 10, wherein the sol-gel transformable material is methyl cellulose.

12. The PCR device of claim 2, wherein the first and second micro-valves form the inlet and outlet of the PCR device, respectively.

13. The PCR device of claim 2, wherein the first micro-valve extends in a direction in which the biochemical fluid is injected into the inlet, and the second micro-valve extends in a direction in which the biochemical fluid is discharged through the outlet.

14. The PCR device of claim 2, wherein the first and second micro-valves are interconnected with the inlet and the outlet, respectively, the first micro-valve branches off from a portion of the PCR channel near the inlet in a different direction from a direction in which the biochemical fluid is injected, and the second micro-valve branches off from a portion of the PCR channel near the outlet in a different direction from a direction in which the biochemical fluid is discharged.

15. The PCR device of claim 2, wherein the first and second micro-valves intersect portions of the PCR channel near the inlet and the outlet of the PCR device, respectively.

16. The PCR device of claim 2, wherein the first and second micro-valves intersect portions of PCR channels of a plurality of PCR devices near inlets and outlets of the PCR devices, respectively.


SEQUENCE LISTING (2 sequences)

SEQ ID NO: 1 - DNA, 22 bp, Artificial Sequence
  forward primer for PCR
cccttgctgagcagatcccgtc

SEQ ID NO: 2 - DNA, 21 bp, Artificial Sequence
  reverse primer for PCR
gggatggtgaagcttccagcc